(19) United States
(12) Patent Application Publication
(10) Pub. No.: US 2007/0042957 A1
(43) Pub. Date: Feb. 22, 2007

(54) TYPE V PHOSPHODIESTERASE INHIBITORS AND NATRIURETIC POLYPEPTIDES

(75) Inventors: John C. Burnett JR., Rochester, MN (US); Horng H. Chen, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research, Rochester, MN (US)

(21) Appl. No.: 11/465,380

(22) Filed: Aug. 17, 2006

Related U.S. Application Data
(60) Provisional application No. 60/709,633, filed on Aug. 19, 2005.

(51) Int. Cl. A61K 38/17; A61K 31/519

(52) U.S. Cl. 514/12; 514/252.16

(57) ABSTRACT

This document provides methods and materials related to PDE V inhibitors, natriuretic polypeptides, and combinations thereof. For example, compositions containing one or more PDE V inhibitors in combination with one or more natriuretic polypeptides are provided herein. In addition, methods for using PDE V inhibitors, natriuretic polypeptides, and combinations thereof to influence heart or renal activities within a mammal are provided herein.

### CROSS-REFERENCE TO RELATED APPLICATIONS

This application claims the benefit of U.S. Provisional Patent Application Ser. No. 60/709,633, filed Aug. 19, 2005.

### STATEMENT AS TO FEDERALLY SPONSORED RESEARCH

This invention was made with government support under grant HL036634-19 awarded by the National Institutes of Health. The government has certain rights in the invention.

### BACKGROUND

1. Technical Field

This document relates to type V phosphodiesterase inhibitors, natriuretic polypeptides, and combinations thereof This document also relates to methods for using type V phosphodiesterase inhibitors, natriuretic polypeptides, and combinations thereof to influence heart or renal activities within a mammal.

2. Background Information

Nesiritide is a recombinant human brain natriuretic peptide (BNP) provided commercially (Scios, Inc.) in the clinical practice to manage acute decompensated heart failure (HF). Type V phosphodiesterase (PDE V) metabolizes cyclic GMP and is abundant in the vasculature and kidney. Sildenafil is a PDE V inhibitor that is used clinically for erectile dysfunction and is undergoing evaluation for managing pulmonary hypertension.

### SUMMARY

This document provides methods and materials related to PDE V inhibitors, natriuretic polypeptides, and combinations thereof. For example, this document provides compositions containing one or more PDE V inhibitors in combination with one or more natriuretic polypeptides. Such compositions can be administered to a mammal such that the mammal experiences improved left ventricular remodeling (e.g., a reduction in left ventricular mass). In addition, such compositions can be administered to a mammal such that the mammal experiences an increase in the renal action of the natriuretic polypeptide to a level that is greater than that observed with a comparable composition lacking PDE V inhibitors. This document also provides methods for using PDE V inhibitors, natriuretic polypeptides, and combinations thereof to improve heart or renal function within a mammal. For example, PDE V inhibitors can be used alone or in combination with a natriuretic polypeptide to reduce left ventricular mass in a mammal.

This document is based, in part, on the discoveries that chronic administration of a PDE V inhibitor results in left ventricular remodeling and that inhibiting PDE V activity enhances the renal effects of administering (e.g., acute subcutaneously administering) an exogenous natriuretic polypeptide (e.g., brain natriuretic peptide; BNP). As described herein, chronic inhibition of PDE V can potentiate endogenous cyclic GMP levels, improve left ventricular remodeling, and enhance the renal actions of exogenous BNP.

In general, this document features a composition comprising, or consisting essentially of, an inhibitor of type V phosphodiesterase and a polypeptide having brain natriuretic peptide activity. The inhibitor can be sildenafil citrate. The polypeptide can be a human brain natriuretic peptide. The polypeptide can be nesiritide. Between about 1 mg and 100 mg of the composition can be the inhibitor. Between about 20 mg and 75 mg of the composition can be the inhibitor. Between about 200 μg and 20 mg of the composition can be the polypeptide. Between about 500 μg and 10 mg of the composition can be the polypeptide.

In another aspect, this document features a method for reducing left ventricular mass in a mammal. The method comprises, or consists essentially of. (a) identifying a mammal in need of reduced left ventricular mass, and (b) administering an inhibitor of type V phosphodiesterase to the mammal under conditions wherein the left ventricular mass of the mammal decreases. The inhibitor can be sildenafil citrate. The method can include administering a polypeptide having brain natriuretic peptide activity to the mammal. The polypeptide can be a human brain natriuretic peptide. The polypeptide can be nesiritide.

In another aspect, this document features a method for increasing the renal action of a polypeptide having brain natriuretic peptide activity. The method comprises, or consist essentially of, administering an inhibitor of type V phosphodiesterase and the polypeptide to a mammal (e.g., a human). The inhibitor can be sildenafil citrate. The polypeptide can be a human brain natriuretic peptide. The polypeptide can be nesiritide. The mammal can be at risk of experiencing heart failure or renal failure. The method can include identifying the mammal as being at risk of experiencing heart failure or renal failure.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains bar graphs plotting the fractional shortening (FS), cardiac output (CO), LV mass, and plasma BNP levels for the group treated with a PDE V inhibitor (PDEVI) and for the untreated CHF group.

FIG. 2 contains bar graphs plotting the glomerular filtration rate (GFR), urinary sodium excretion (UNaV), and proximal tubule fractional sodium absorption (PTFNa) levels for the group treated with a PDEVI and for the untreated CHF group, each treated either with (black bars) or without (white bars) BNP.

### DETAILED DESCRIPTION

This document provides methods and materials related to PDE V inhibitors, natriuretic polypeptides, and combinations thereof. For example, this document provides compositions containing one or more PDE V inhibitors, one or more polypeptides having natriuretic polypeptide activity, or combinations thereof. The term “PDE V inhibitor” as used herein refers to any compound having the ability to reduce the activity of a PDE V polypeptide within a cell. The reduction can be any level of reduction including, without limitation, a 10, 20, 30, 40, 50, 60, 70, 80, 90, 95, or 100 percent reduction in the activity of a PDE V polypeptide. Examples of PDE V inhibitors include, without limitation, sildenafil citrate, sodium 1-[6-chloro-4-(3,4-methylenedioxybenzyl)-aminoquinazolin-2-yl]piperidine-4-carboxylate sesquihydrate (E4021), tadalafil, vardenafil, and zaprinast. Examples of polypeptides having natriuretic polypeptide activity (e.g., BNP activity) include, without limitation, BNP polypeptides, C-type natriuretic peptide (CNP), urodilatin, snake natriuretic peptide (DNP), and atrial natriuretic peptide (ANP) polypeptides. BNP polypeptides can include alternatively spliced forms of BNP such as those described in U.S. patent application Ser. No. 10/561,014. For example, a BNP2 or BNP3 polypeptide can be used. In some cases, a polypeptide such as any one of the polypeptides described in U.S. Provisional Patent Application No. 60/836,581 can be used.

The polypeptides having natriuretic polypeptide activity can have a non-naturally occurring sequence or can have a sequence present in any species (e.g., human, horse, pig, goat, cow, dog, cat, rat, or mouse). For example, a polypeptide having natriuretic polypeptide activity can be a human BNP polypeptide having one or more amino acid changes. In some cases, a polypeptide having natriuretic polypeptide activity can be a human BNP polypeptide such as nesiritide. A polypeptide having natriuretic polypeptide activity can contain one or more modifications. For example, a polypeptide having natriuretic polypeptide activity can be modified to be pegylated or to contain additional amino acid sequences such as an albumin sequence (e.g., a human albumin sequence). In some cases, a polypeptide having natriuretic polypeptide activity can be a fusion polypeptide that contains a fragment of an albumin sequence (e.g., a human albumin sequence). In some cases, a polypeptide having natriuretic polypeptide activity can be covalently attached to oligomers, such as short, amphiphilic oligomers that enable oral administration or improve the pharmacokinetic or pharmacodynamic profile of a conjugated polypeptide having natriuretic polypeptide activity. The oligomers can comprise water soluble PEG (polyethylene glycol) and lipid soluble alkyls (short chain fatty acid polymers). See, for example, International Patent Application Publication No. WO 2004/047871. In some cases, a polypeptide having natriuretic polypeptide activity can be fused to the Fc domain of an immunoglobulin molecule (e.g., an IgG1 molecule) such that active transport of the fusion polypeptide across epithelial cell barriers via the Fe receptor occurs.

The compositions provided herein can contain one or more than one (e.g., two, three, four, or more) PDE V inhibitors without containing a polypeptide having natriuretic polypeptide activity. In some cases, the compositions provided herein can contain one or more than one (e.g., two, three, four, or more) PDE V inhibitor as well as one or more than one (e.g., two, three, four, or more) polypeptide having natriuretic polypeptide activity. For example, a composition can contain sildenafil citrate and nesiritide. In some cases, a composition provided herein can contain sildenafil citrate, tadalafil, and nesiritide. The compositions provided herein can contain any amount of a PDE V inhibitor. For example, a composition can contain between 1 mg and 1 g of a PDE V inhibitor (e.g., between 5 and 500 mg, between 10 and 250 mg, between 20 and 100 mg, or between 25 and 50 mg of a PDE V inhibitor) such as sildenafil citrate. Likewise, the compositions provided herein can contain any amount of a polypeptide having natriuretic polypeptide activity. For example, a composition can contain between 50 μg and 500 mg of a polypeptide having natriuretic polypeptide activity (e.g., between 100 μg and 200 mg, between 200 μg and 100 mg, between 500 μg and 10 mg, or between 700 μg and 7 mg of a polypeptide having natriuretic polypeptide activity) such as nesiritide. In some cases, a composition can contain between about 25 mg and 50 mg of a PDE V inhibitor and between about 0.7 mg and 7 mg of a polypeptide having natriuretic polypeptide activity. The dose supplied by a composition (e.g., capsule, pill, or tablet) can vary since an effective amount can be reached by administrating either one or multiple units (e.g., capsules, pills, or tablets).

Any method can be used to formulate a composition provided herein. For example, common formulation mixing and preparation techniques can be used to make a composition having the components described herein. In addition, the compositions provided herein can be in any form. For example, a composition provided herein can be in the form of a solid, liquid, and/or aerosol including, without limitation, powders, crystalline substances, gels, solutions, suspensions, partial liquids, sprays, pills, capsules, tablets, and gelcaps. Typically, a composition containing one or more PDE V inhibitors, one or more polypeptides having natriuretic polypeptide activity, or combinations thereof can be prepared for oral administration by mixing the components with one or more of the following: a filler, a binder, a disintegrator, a lubricant, and a coloring agent. Lactose, corn starch, sucrose, glucose, sorbitol, crystalline cellulose, silicon dioxide, or the like can be used as the filler. Polyvinyl alcohol, polyvinyl ether, ethyl cellulose, methyl cellulose, gelatin, hydroxypropyl cellulose, hydroxypropylmethyl cellulose, calcium citrate, dextrin, or pectin can be used as the binder. Magnesium stearate, talc, polyethylene glycol, silica, or hardened plant oil can be used as the lubricant. A pharmaceutically acceptable coloring agent can be used as the coloring agent.

A composition (e.g., pill or tablet) containing one or more PDE V inhibitors, one or more polypeptides having natriuretic polypeptide activity, or combinations thereof can be formulated to contain additional components such as pharmaceutically acceptable aqueous vehicles or pharmaceutically acceptable solid vehicles, Examples of pharmaceutically acceptable aqueous vehicles include, without limitation, saline, water, and acetic acid. Typically, pharmaceutically acceptable aqueous vehicles are sterile. Any well known pharmaceutically acceptable material such as gelatin and cellulose derivatives can be used as a pharmaceutically acceptable solid vehicle. In addition, a pharmaceutically acceptable solid vehicle can be a solid carrier including, without limitation, starch, sugar, or bentonite. Further, a composition can be made using conventional procedures that employ solid carriers, lubricants, and the like.

A capsule, tablet, or particle containing one or more PDE V inhibitors, one or more polypeptides having natriuretic polypeptide activity, or combinations thereof can be covered with an enteric coating (e.g., a polymer) effective for shielding the capsule, tablet, or particle from digestion during transit through the upper portions of the digestive tract. An enteric coating can be a cellulose- or acrylic-based coating. An example of a cellulose-based coating is a cellulose acetate phthalate (CAP) coating, such as Aquacoat (FMC BioPolymer, Philadelphia, Pa.). A coating can dissolve when it reaches the neutral pH of the upper small intestine.

In some cases, a composition containing one or more polypeptides having natriuretic polypeptide activity, one or more PDE V inhibitors, or combinations thereof can be formulated for oral administration using a microencapsulation technique. For example, any combination of one or more polypeptides having natriuretic polypeptide activity and one or more PDE V inhibitors can be mixed with a stabilizing agent in an aqueous solution. The solution can be coated onto edible beads, e.g., nonpareils, and microencapsulated with a water emulsifiable enteric coating composition. The stabilizing agent can be any agent that protects a therapeutic polypeptide from denaturation during the encapsulation process. See, for example, U.S. Pat. No. 6,613,332. In some cases, microcapsules of chitosan-alginate modified with excipients such as HPMCAS, talc, microcrystalline cellulose, polymethacrylates, and/or pectins can be used to formulate a composition provided herein. In addition, carriers including, without limitation, hydrogels, nanoparticles, and liposomes, can be used.

In some cases, a composition containing one or more polypeptides having natriuretic polypeptide activity, one or more PDE V inhibitors, or combinations thereof can be formulated for parenteral administration, particularly in the form of liquid solutions or suspensions in aqueous physiological buffer solutions. Formulations for parenteral administration may contain excipients suitable for injection into a mammal (e.g., a human), including sterile water or saline, oils of vegetable origin, hydrogenated naphthalenes, ammonium acetate, benzalkonium chloride, benzethonium chloride, benzyl alcohol, Brij 35, Brij 97, calcium gluceptate, chlorobutanol, polyoxyethylated castor oil, deoxycholate, citric acid monohydrate, diethanolamine, ethanol, gamma cyclodextrin, glycerin, lactobionic acid, lysine, magnesium chloride, mannitol, methylparaben, polyalkylene glycols, polyethylene glycol, PEG 1000, PEG 300, PEG 3350, PEG 400, PEG 600, polyethylene glycol 40 stearate, poloxamer 188, poloxamer 237, poloxamer 338, poloxmer 407, polyoxyethylene 100 stearate, polyoxyethylene 40 stearate, polyoxyethylene 50 stearate, polysorbate 20, polysorbate 80, povidone, propylene glycol, saccharin sodium, sodium acetate, sodium citrate dehydrate, sodium deoxycholate, sodium benzoate, and sodium tartrate. In particular, biocompatible, biodegradable lactide polymer, lactide/glycolide copolymer, or polyoxyethylene-polyoxypropylene copolymers are examples of excipients for controlling the release of a compound in vivo. Other suitable parenteral delivery systems include ethylene-vinyl acetate copolymer particles, implantable devices such as osmotic pumps or other implantable infusion systems, mucosal delivery systems, and liposomes.

In some cases, a composition containing one or more polypeptides having natriuretic polypeptide activity, one or more PDE V inhibitors, or combinations thereof can be a powder (e.g., a lyophilized powder). Such a powder can be reconstituted prior to administration (e.g., parenteral administration). The powder can be reconstituted with a diluent, such as a preservative-free diluent. Examples of such diluents include 5% Dextrose Injection (D5W), USP; 0.9% Sodium Chloride Injection, USP; 5% Dextrose and 0.45% Sodium Chloride Injection, USP; and 5% Dextrose and 0.2% Sodium Chloride Injection, USP.

Any method can be used to obtain PDE V inhibitors and polypeptides having natriuretic polypeptide activity. In some cases, PDE V inhibitors and polypeptides having natriuretic polypeptide activity can be obtained using common chemical or biochemical extraction, isolation, or synthesis techniques. Polypeptides having natriuretic polypeptide activity can be produced using standard recombinant DNA techniques. For example, recombinant DNA technology can be used with an expression system, such as an E. coli expression system, to produce polypeptides having natriuretic polypeptide activity. The polypeptides can be isolated, e.g., from inclusion bodies solubilized in about 8 M urea, and can be purified using standard chromatographic techniques. In some cases, the components of the compositions provided herein can be obtained commercially. For example, sildenafil citrate can be obtained from Pfizer, Inc., and nesiritide can be obtained from Scios Inc.

This document also provides methods and materials related to improving heart or renal function in a mammal (e.g., human, horse, pig, goat, cow, dog, cat, rat, or mouse). As described herein, compositions containing one or more PDE V inhibitors can be used to induce left ventricular remodeling within a mammal. In addition, compositions containing one or more PDE V inhibitors and one or more polypeptides having natriuretic polypeptide activity can be used to increase renal function (e.g., glomerular filtration rate or urinary sodium excretion) within a mammal. In some cases, compositions containing one or more PDE V inhibitors and one or more polypeptides having natriuretic polypeptide activity can be administered to a mammal such that the mammal experiences an increase in the renal action of the natriuretic polypeptide to a level that is greater than that observed with a comparable composition lacking PDE V inhibitors. In some embodiment, the compositions provided herein can be used to treat mammals having a heart or renal condition (e.g., heart failure, hypertension, coronary artery disease, or renal failure) or mammals at risk of experiencing a heart or renal condition.

Any route of administration (e.g., oral or parenteral administration) can be used to administer a composition provided herein to a mammal. For example, a composition provided herein can be administered orally or parenterally (e.g., a subcutaneous, intraperitoneal, intramuscular, intraorbital, intracapsular, intraspinal, intrastemal, or intravenous injection).

Before administering a composition provided herein to a mammal, the mammal can be assessed to determine whether or not the mammal has a heart or renal condition or is at risk of experiencing a heart or renal condition. Any method can be used to determine whether or not a mammal has a heart or renal condition. For example, a mammal (e.g., human) can be identified as having a heart or renal condition using standard diagnostic techniques such as techniques that analyze heart function or kidney function. In addition, diagnostic methods such as reviewing an individual's prior medical conditions and treatments, interviewing and evaluating an individual, and collecting and analyzing biological samples (e.g., blood samples) from an individual can be used to identify the presence of a heart or renal condition or to determine the likelihood of experiencing a heart or renal condition.

In identifying a heart condition, an individual can be questioned about dyspnea, cough, nocturia, generalized fatigue and other signs and symptoms of a heart condition. An individual also can be asked about chest pain, hypertension, myocardial infarction, and a family history of a heart condition. A physical examination can include an assessment of an individual's general appearance for evidence of resting dyspnea, cyanosis, and cachexia. The individual's blood pressure and heart rate can be determined. A physical examination also can include exercise testing, monitoring an individual for evidence of periodic breathing (Cheyne-Stokes respiration), assessing jugular venous distention to determine whether or not jugular venous pressure is elevated, and locating the point of maximal impulse of the left ventricle as a means for evaluating the size of the heart. In addition, an individual can be evaluated for lower extremity edema, a common sign of heart failure, and hepatomegaly, which may occur because of right-sided heart failure and venous congestion. Standard diagnostic tests including, without limitation, electrocardiography, chest radiography, echocardiography, angiography, positron emission tomography, MRI, and ultrafast and cine computed tomography (CT) also can be used to identify a heart condition.

Analysis of biological samples (e.g., blood samples) in the identification of a heart condition can include determining noradrenaline, renin, angiotensin II, and/or aldosterone levels. In addition, the presence, absence, or level of one or more than one polypeptide having natriuretic activity (e.g., ANP, BNP, BNP2, or BNP3) can be detected in a biological sample from a mammal, and the mammal can be classified as having a heart condition or not having a heart condition based, at least in part, on the presence, level, or absence of the one or more than one polypeptide having natriuretic activity. Polypeptides having natriuretic activity can increase early in the course of cardiac dysfunction, prior to the onset of symptoms. In some cases, the level of a polypeptide having natriuretic activity can be determined and compared to the level of the polypeptide from a control population (e.g., the average level of the polypeptide from a plurality of control subjects without heart conditions). The presence of a polypeptide having natriuretic activity or an increase in the level of a polypeptide having natriuretic activity relative to that of a control population can be indicative of a heart condition. In some cases, an N-terminal polypeptide of a polypeptide having natriuretic activity (e.g., N-terminal-ANP) can reflect the presence and severity of ventricular dysfunction more accurately than the polypeptide having natriuretic activity (e.g., ANP). In some cases, the presence, absence, or level of one or more than one ribonucleic acid encoding a polypeptide having natriuretic activity can be detected in a biological sample from a mammal, and the mammal can be classified as having or not having a heart condition based, at least in part, on the presence, level, or absence of the one or more than one ribonucleic acid encoding a polypeptide having natriuretic activity. A polypeptide (e.g., a polypeptide having natriuretic activity) or a ribonucleic acid (e.g., a ribonucleic acid encoding a polypeptide having natriuretic activity) can be detected using well known techniques including, without limitation, ELISA, surface plasmon resonance, mass spectrometry, and PCR.

Additional factors that can be considered in identifying heart conditions can include, for example, genetic factors, such as SNPs, and the levels of other cardiac markers such as troponin I or T, high sensitive C-reactive protein (hs-CRP), creatinine kinase (CK), CK-MB, creatinine, or myoglobin. In general, myoglobin is not cardiac specific, but is released from infarcted myocardium at an early stage (about 2-3 hours post infarction) and returns to normal within about 24 hours. Cardiac isoforms of troponin I and troponin T are specific, but appear in the circulation later than myoglobin (5 to 48 hours post infarction). Myocardial tissue contains one isoform of CK-MB, while skeletal tissue has different isoforms. Antibodies having specific binding affinity for such cardiac markers are available commercially.

Identifying a kidney condition in an individual can be based on the history, physical examination, and laboratory evaluation, as described in standard texts and reviews. See, e.g., Remuzzi et al., N Engl J Med, 346:1145-51 (2002) and Levey, N Engl J Med, 347:1505-11 (2002). For example, signs and symptoms of a kidney condition can include, without limitation, high blood pressure, unexplained weight loss, anemia, nausea or vomiting, malaise, fatigue, bloody or tarry stools, a yellowish-brown cast to the skin, persistent itching, symptoms during urination, skin rash, and a family history of kidney conditions. In some cases, the level of kidney function can be assessed by determining glomerular filtration rate (GFR). For example, GFR can be estimated from serum creatinine levels by using prediction equations that also take into account age, sex, race, and body size. Two such equations are the Cockcrofl-Gault equation (Cockerofl and Gault, Nephron, 16:31-41, 1976)) and the Abbreviated Modification of Diet in Renal Disease (MDRD) study equation (Levey et al., Ann Intem Med, 130:461-70 (1999); Levey et al., J Am Soc Nephrol, 11:0828 (2000)). The normal GFR in young adults can be about 120 to 130 mL/min per 1.73 m² and can decline with age. A GFR level less than 60 mL/min per 1.73 m² can represent a loss of half or more of the adult level of normal kidney function. In addition to GFR, another indicator of kidney function is blood urea nitrogen (BUN).

Urinalysis to detect and monitor proteinuria also can be useful in identifying a kidney condition. Mammals without a kidney condition usually excrete very small amounts of protein in the urine. Increased excretion of protein can be a sensitive marker for a kidney condition. For example, an albumin-creatinine ratio greater than 30 mg/g in untimed (spot) urine samples can be indicative of a kidney condition. In some cases, an albumin-creatinine ratio greater than 17 mg/g in men and greater than 25 mg/g in women can be indicative a kidney condition. Other markers of a kidney condition include abnormalities in urine sediment, abnormalities in blood and urine chemistry measurements such as electrolyte levels, and abnormal findings on imaging studies (e.g., ultrasound imaging or magnetic resonance imaging studies).

After identifying a mammal as having a heart or renal condition or as being at risk of experiencing a heart or renal condition, the mammal can be treated with a composition provided herein. For example, a composition containing one or more PDE V inhibitors and one or more polypeptides having natriuretic polypeptide activity can be administered to a mammal. In some cases, a composition containing one or more than one PDE V inhibitor and a composition containing one or more than one polypeptide having natriuretic polypeptide activity can be administered to a mammal. The route of administration of each composition can be the same or can be different. In addition, each composition can be administered at the same time or at a different time. In some cases, a composition containing one or more than one PDE V inhibitor can be administered in the absence of administration of a composition containing one or more than one polypeptide having natriuretic polypeptide activity.

A composition provided herein, e.g., a composition containing one or more PDE V inhibitors and one or more polypeptides having natriuretic polypeptide activity, can be administered to a mammal in any amount, at any frequency, and for any duration effective to achieve a desired outcome (e.g., to reduce left ventricular mass or to increase renal function). Any method can be used to determine whether or not the mammal experiences a reduction in left ventricular mass or an increase in renal function including, without limitation, those methods described herein. In some cases, left ventricular mass or renal function can be assessed prior to treatment. After treatment with a composition provided herein, left ventricular mass or renal function can be determined again, and the results compared to those obtained before treatment.

An effective amount of a composition can be any amount that improves heart or renal function within a mammal without producing significant toxicity to the mammal. In some cases, a commonly prescribed amount of a PDE V inhibitor or polypeptide having natriuretic activity can be used. For example, the amount of sildenafil can be 25 mg, 50 mg, or 100 mg per day, the amount of vardenafil can be 10 mg or 20 mg per day, and the amount of nesiritide can be an IV bolus of 2 μg/kg followed by a continuous infusion of 0.01 μg/kg/min for one day, two days, or more than two days. In some cases, a commonly prescribed amount can be used to estimate an effective dose. If a particular mammal fails to respond to a particular amount, then the amount can be increased by, for example, 1.1, 1.2, 1.3, 1.4, 1.5, 1.6, 1.7, 1.8, 1.9, 2, 3, 4, 5, 6, 7, 8, 9, or 10 fold. For example, the infusion amount of nesiritide can be increased by 0.005 μg/kg/min (preceded by a bolus of 1 μg/kg) every three hours up to a dose of 0.03 μg/kg/min. In some cases, an increase in the amount of a polypcptide having natriuretic activity can be accompanied by an increase in the amount of a PDE V inhibitor. In other cases, the amount of a PDE V inhibitor can remain constant while the amount of a polypeptide having natriuretic activity is increased. After receiving an increased amount of a composition, the mammal can be monitored for both responsiveness to the treatment and toxicity symptoms, and adjustments made accordingly. The effective amount can remain constant or can be adjusted as a sliding scale or variable dose depending on the mammal's response to treatment. The dose of a PDE V inhibitor and the dose of a polypeptide having natriuretic activity can be increased or decreased simultaneously, the dose of one can be increased while the other is decreased, or the dose of one can remain constant while the other is varied.

Various factors can influence the actual effective amount used for a particular application. For example, the frequency of administration, duration of treatment, use of multiple PDE V inhibitors or polypeptides having natriuretic polypeptide activity within a single composition, route of administration, and severity of the heart or renal condition may require an increase or decrease in the actual effective amount administered.

The frequency of administration can be any frequency that improves heart or renal function within a mammal without producing significant toxicity to the mammal. For example, the frequency of administration can be from about four times a day to about once every other month, or from about once a day to about once a month, or from about once every other day to about once a week. In addition, the frequency of administration can remain constant or can be variable during the duration of treatment. As with the effective amount, various factors can influence the actual frequency of administration used for a particular application. For example, the effective amount, duration of treatment, use of multiple PDE V inhibitors or polypeptides having natriuretic polypeptide activity within a single composition, route of administration, and severity of the heart or renal condition may require an increase or decrease in administration frequency.

An effective duration of administration can be any duration that improves heart or renal function within a mammal without producing significant toxicity to the mammal. The effective duration can vary from several days to several weeks, months, or years. In general, the effective duration for improving heart or renal function can range in duration from several days to several months. Once the administrations are stopped, however, a heart or renal condition may return. Thus, the effective duration for preventing the return of a heart or renal condition can be in some cases for as long as an individual mammal is alive. Typically, an effective duration can range from about one to two weeks to about 36 months. Again, prophylactic treatments can be typically longer in duration and can last throughout an individual mammal's lifetime.

Multiple factors can influence the actual effective duration used for a particular treatment or prevention regimen. For example, an effective duration can vary with the frequency of administration, amount administered, use of multiple PDE V inhibitors or polypeptides having natriuretic polypeptide activity within a single composition, route of administration, and severity of the heart or renal condition.

After administering a composition provided herein to a mammal, the mammal can be monitored to determine whether or not heart or renal function has been improved. For example, a mammal can be assessed after treatment to determine whether or not left ventricular mass decreased or renal function increased. As described herein, any method can be used to assess improvements in heart or renal function. For example, a mammal's baseline level of a polypeptide having natriuretic activity before treatment can be compared to the level of the polypeptide at various time points after treatment (e.g., one or more hours, days, weeks, or months after treatment). A decrease in the level of the polypeptide having natriuretic activity relative to the baseline level is indicative of an improvement in heart function. In some cases, an echocardiogram obtained before treatment can be compared to an echocardiogram obtained at one or more than one time point during and/or after treatment to determine whether or not left ventricular mass has been reduced. In some cases, GFR can be assessed before and aifer treatment to determine whether or not renal function has improved.

The invention will be further described in the following examples, which do not limit the scope of the invention described in the claims.

### EXAMPLES

### Example 1

### PDE V Inhibition has Favorable Effects on Left Ventricular (LV) Remodeling and Potentiates the Renal Actions of Exogenous BNP in a Congestive Heart Failure Model

Studies were conducted in 2 groups of male mongrel dogs (18-23 kg) with overt chronic heart failure produced by rapid ventricular pacing at 240 beats per minute (bpm) for ten days on a fixed sodium diet. Group 1 (n=6) received Sildenafil (50 mg orally three times a day) during the 10 days of pacing and one dose an hour before the subcutaneous (SQ) administration of BNP on day 11. Group 2 (n=6) received no PDE V inhibitor, but did receive SQ BNP alone on day 11 of CHF.

A model of pacing-induced overt chronic heart failure was used (Chen et al., Circulation, 100:2443-2448 (1999)). Briefly, all dogs underwent implantation of a programmable cardiac pacemaker (Medtronic, Minneapolis, Minn.). Under pentobarbital sodium anesthesia (30 mg/kg, intravenous) and artificial ventilation (Harvard respirator, Harvard Apparatus, Millis, Mass.) with 5 L/min supplemental oxygen, a left lateral thoracotomy and pericardiotomy were performed. With the heart exposed, a screw-in epicardial pacemaker lead was implanted into the right ventricle. The pacemaker generator was implanted subcutaneously into the left chest wall and connected to the pacemaker lead. Dogs received pre- and post-operative prophylactic antibiotic treatment with 225 mg clindamycin subcutaneously and 400,000 U procaine penicillin G plus 500 mg dihydrostreptomycin intramuscularly (Combiotic, Pfizer, Inc., New York, N.Y.). Post-operative prophylactic antibiotic was continued through the first two post-operative days. Dogs were fed a fixed sodium diet (58 mEq/day, Hill's ID) and allowed water ad lib. All dogs were walked daily. Appetite, activity, body temperature, and condition of surgical skin sites were documented. Following a 14-day post-operative recovery period, the pacemaker was turned on at 240 bpm. Group 1 (n=6) received Sildenafil (50 mg orally three times a day) during the 10 days of pacing, and Group 2 (n=5) received no PDE V inhibitor.

Echocardiography was carried out at baseline before the pacemaker was turned on and at day 10 of pacing to determine LV fractional shortening (FS). Echocardiographic images were obtained in the conscious dog standing in a minimally restraining sling. Simultaneous electrocardiography was obtained. The optimal images were generally obtained from the right lateral chest wall just above the sternum at the equivalent of the anterior axillary line. Images analogous to the para-sternal long axis and para-sternal short axis views (human echocardiography) were consistently obtained at the base, mid-ventricular, and para-apical levels. LV two-dimensional (2D) and M-mode images were obtained with a 2.5-MHz ultrasonic transducer (General Electric Systems Five). LV M-mode tracings were obtained under 2D guidance using the 2D short-axis views between the two papillary muscles just distal to the mitral valve leaflet tips. All images were obtained in sinus rhythm. Care was taken to ensure that the M-mode cursor transects the LV in the midline, that the LV image is “on axis” (LV is round, not oval indicating an image between short and long axes), and that the cursor is perpendicular to the LV wall detecting maximal LV thickening of both the septal and posterior walls. End-diastole was defined as the maximal LV diastolic diameter at the peak of the R wave on the EKG, and end-systole was defined as the peak of the LV posterior wall motion. M-mode measurements were made in accordance with the American Society of Echocardiography leading-edge convention using the steepest continuous endocardial echoes. Measurements included LV end-diastolic (LVDd), and end-systolic dimensions (LVDs) were averaged for average dimension. FS (%)=[(LVDd-LVSd)/LVDd]*100.

Acute SQ BNP administration was performed as follows. On day 11 of rapid ventricular pacing at 240 bpm, an experiment was carried out to determine the cardiorenal and humoral function in both groups and also the response to acute SQ BNP administration (5 μg/kg). On the night before experimentation, animals were fasted and given 300 mg of lithium carbonate for assessment of renal tubular function. On the morning of the experiment, only group 1 received a final dose of Sildenafil 50 mg one hour before the dogs were anesthetized with sodium pentobarbital (15 mg/kg, i.v.), intubated, and mechanically ventilated with supplemental oxygen (Harvard respirator, Amersham, Millis, Mass.) at 20 cycles per minute. A flow-directed balloon-tipped thermodilution catheter (Ohmeda, Criticath, Madison, Wis.) was advanced into the pulmonary artery via the external jugular vein for cardiac hemodynamic measurement. The femoral artery was cannulated for blood pressure monitoring and blood sampling. The femoral vein was also cannulated for insulin and normal saline infusion. The left kidney was exposed via a flank incision, and the ureter was cannulated for urine collection. A calibrated electromagnetic flow probe was placed around the renal artery to measure renal blood flow (RBF).

The experiment began after a 60-minute equilibration period, with a 30-minute baseline urinary clearance. After the 30-minute baseline urinary clearance, SQ canine BNP 5 μg/kg was administered in the right hind leg. Following a 15-minute lead-in period, a 60-minute urinary clearance period was performed. Cardiovascular parameters measured during the acute experiment included mean arterial blood pressure (MAP), right atrial pressure (RAP), mean pulmonary arterial pressure (PAP), cardiac output (CO), and pulmonary capillary wedge pressure (PCWP). CO was determined by thermodilution in triplicate and averaged (Cardiac Output model 9510-A computer, American Edwards Laboratories, Irvine, Calif.). MAP was assessed via direct measurement from the femoral arterial catheter. Systemic vascular resistance (SVR) was calculated as [SVR=(MAP-RAP)/CO]. Insulin was administered intravenously at the start of the equilibration period as a calculated bolus, followed by a 1 mL/min continuous infusion to achieve plasma levels of 40-60 mg/dL. Glomerular filtration rate (GFR) was measured by insulin clearance.

Cardiovascular hemodynamics were measured at the start of each urinary clearance. Arterial blood was collected in heparin and EDTA tubes and immediately placed on ice midway through each clearance. After centrifugation at 2,500 rpm at 4° C., plasma was decanted and stored at −20° C. until analysis. Urine was collected on ice during the entire period of each clearance for assessment of urine volume, electrolytes, and insulin. Urine collected for cGMP analysis was heated to more than 90° C. before storage.

At the end of the acute experiment, the heart was harvested, and the LV was carefully dissected out and weighed to determine LV mass. The LV mass was divided by the body weight to give the LV mass/Kg body weight.

Plasma samples for BNP renin and angiotensin II were measured by radioimmunoassay (RIA) using methods described elsewhere (Chen et al., Circulation, 96:1-524 (1997)). Plasma and urinary samples for cGMP were measured by RIA using methods described elsewhere (Steiner et al., J. Biol. Chem., 247:1106-1113 (1972)). Urinary and plasma insulin concentrations were measured by the anthrone method (Davidson and Sachner, J. Lab. Clin. Med., 62:351-356 (1963)). Urinary and plasma lithium levels were determined by flame emission spectrophotometry (model 357, Instrumentation Laboratory, Wilmington, Mass.). Employing the lithium clearance (CLLi) technique, distal fractional reabsorption of sodium (DFRNa) was calculated utilizing the following equation: DFRNa=(CL_{Li}−CL_{Na})/CL_{Li}×100; where CL_{Li}=(Urine Li×Urine flow)/Plasma Li, and CL_{Na}=(Urine Na×Urine flow)/Plasma Na.

Results were expressed as mean ±SEM. Comparisons within the group were made by paired Student's t test, and comparison between groups by unpaired Student's t test. GraphPad Prism software was used for the above calculation. Statistical significance was accepted as p<0.05.

Results

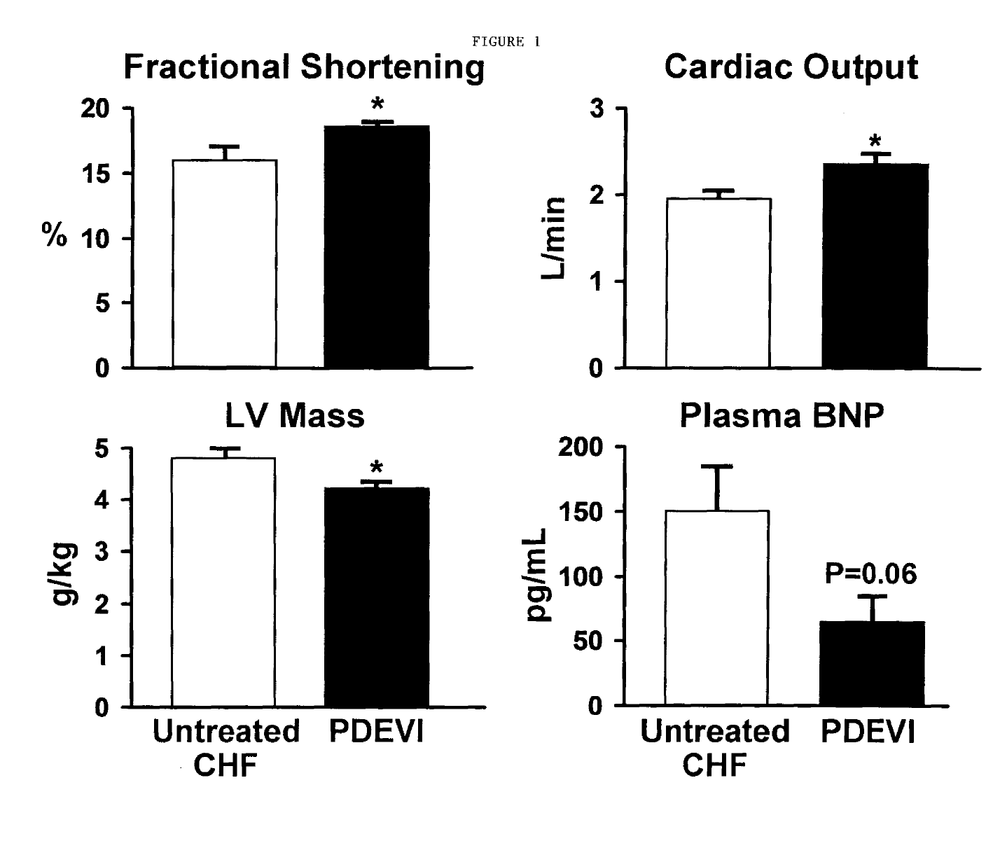

10 days of treatment with the PDE V inhibitor resulted a reduction in LV mass as compared to the LV mass observed in the untreated group (FIG. 1). This was associated with an improvement of LV FS and CO as compared to the untreated group (FIG. 1). Furthermore, there was a strong trend for plasma BNP levels to be lower in the PDE V inhibitor-treated group as compared to the untreated group (FIG. 1). The cardiorenal and neurohumoral profile was determined for the PDE V inhibitor-treated and untreated groups (Table 1). MAP and cardiac filling pressures were similar between the two groups. UNaV, UV, and RBF were similar between the two groups, while there was a non-significant trend for GFR to be higher in the PDE V inhibitor-treated group. Both plasma cGMP and urinary cGMP excretion were significantly higher in the PDE V inhibitor-treated group as compared to the untreated group.

TABLE 1
Cardiorenal and Neurohumoral Profile

| | PDE V inhibitor- | |
|---|---|---|
| | Treated | Untreated |
| MAP, mmHg | 110 ± 4 | 110 ± 5 |
| PAP, mmHg | 27 ± 1 | 28 ± 2 |
| PCWP, mmHg | 23 ± 1 | 22 ± 2 |
| RAP, mmHg | 11 ± 1 | 9 ± 1 |
| GFR, mL/min | 30 ± 5† | 25 ± 6 |
| RBF, mL/min | 160 ± 21 | 160 ± 22 |
| UNaV, μEq/min | 2 ± 1 | 3 ± 1 |
| UV, mL/min | 0.12 ± 0.01 | 0.11 ± 0.03 |
| Plasma cGMP pmol/mL | 43 ± 2* | 21 ± 3 |
| Urinary cGMP pmol/min | 4219 ± 900* | 2000 ± 300 |

Mean ± SE;
MAP: mean arterial blood pressure;
CO: cardiac output;
PAP: mean pulmonary arterial pressure;
PCWP: pulmonary capillary wedge pressure;
RAP: right atrial pressure;
*P < 0.05 versus Untreated,
†P = 0.5 versus Untreated

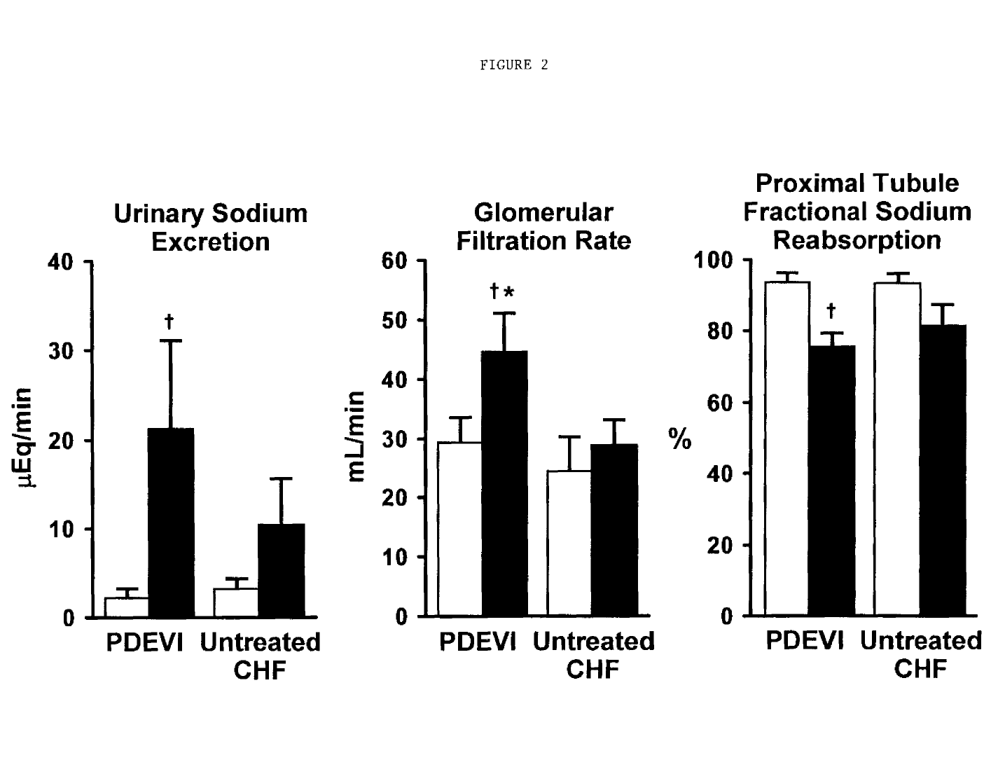

In the PDE V inhibitor-treated group, SQ BNP resulted in an increase in GFR that was not observed in the untreated group (FIG. 2). UNaV was increased, and PTFNa decreased significantly with SQ BNP in the PDE V inhibitor-treated group, while it only trended to change in the untreated group (FIG. 2).

With the administration of SQ BNP, plasma BNP levels increased similarly in both groups. However, both plasma cGMP and urinary cGMP excretion were significantly higher in the PDE V inhibitor-treated group as compared to the untreated group (Table 2). Both plasma angiotensin II and renin decreased significantly with SQ BNP in the PDE V inhibitor-treated group, but were unchanged in the untreated group (Table 2). In both the PDE V inhibitor-treated and untreated groups, PCWP and PAP decreased with SQ BNP administration. (Table 2). MAP was significantly reduced in the PDE V inhibitor-treated group, while it remained unchanged in the untreated group. CO was unchanged in both groups with SQ BNP.

TABLE 2
Humoral and hemodynamic response to acute SQ BNP

| | Baseline | SQ BNP |
|---|---|---|
| PDE V inhibitor-treated | | |
| Group | | |
| Plasma cGMP (pmol/mL) | 43 ± 2 | 46 ± 3† |
| Urinary cGMP Excretion | 4219 ± 900 | 8600 ± 1600*† |
| (fmol/min) | | |
| Plasma Renin | 16 ± 2 | 9 ± 2* |
| Plasma Ang II | 124 ± 2 | 37 ± 2* |
| MAP | 110 ± 4 | 103 ± 7* |
| PAP | 27 ± 1 | 23 ± 1* |
| RAP | 11 ± 1 | 8 ± 1* |
| PCWP | 23 ± 1 | 18 ± 1* |
| Untreated CHF Group | | |
| Plasma cGMP (pmol/mL) | 21 ± 3 | 25 ± 3 |
| Urinary cGMP Excretion | 2000 ± 300 | 3580 ± 351* |
| (fmol/min) | | |
| Plasma Renin | 21 ± 23 | 20 ± 5 |
| Plasma Ang II | 72 ± 17 | 73 ± 31 |
| MAP | 110 ± 4 | 110 ± 5 |
| PAP | 28 ± 1 | 25 ± 1* |
| RAP | 9 ± 1 | 6 ± 1* |
| PCWP | 22 ± 12 | 20 ± 1* |

Mean ± SE;
*P < 0.05 versus Baseline.
†P < 0.05 versus Untreated

The results provided herein demonstrate that 10 days of PDE V inhibitor treatment resulted in improved LV remodeling and improved systolic function as demonstrated by a decrease in LV mass, increased FS and CO with a strong trend for reduction in plasma BNP. Furthermore, PDE V inhibitor treatment potentiated the renal effects of exogenous BNP resulting in an increase in GFR and UNaV that was not observed in the untreated group.

The improvement of LV remodeling with the PDE V inhibitor in this experimental model was associated with an improvement of CO despite the persistence of an increase in cardiac filling pressures. Despite the continuing RV pacing in the model that resulted in increased filling pressures, PDE V inhibitor treatment attenuated the adverse LV remodeling resulting in decreased LV mass, increased LV FS and CO with a strong trend in the decrease of endogenous BNP production. The mechanisms of this improvement in LV remodeling are likely related to the potentiation of endogenous cGMP as demonstrated by the increased plasma cGMP.

Urinary sodium excretion and urine flow were similar between the PDE V inhibitor-treated and untreated groups with a trend for greater GFR in the PDE V inhibitor-treated group. These results were surprising in view of the improved LV function in the PDE V inhibitor-treated group. This may be explained by the fact that as a result of the continued RV pacing in this model, the cardiac filling pressures were similar between the PDE V inhibitor-treated and untreated groups, and therefore renal venous congestion did not improve and hence the lack of significant improvement of renal function.

With the administration of exogenous BNP, there was an increase in GFR that was not observed in the untreated group. Furthermore, UNaV was increased, and PTFNa decreased significantly with SQ BNP in the PDE V inhibitor-treated group, while it only trended to change in the untreated group. This improvement in renal response to exogenous BNP in the PDE V inhibitor-treated group was associated with a much greater UcGMPV, a marker of the enhanced renal action of BNP. The renal enhancing action of the natriuretic peptides can be attenuated in overt CHF and can be due, in part, to the increased cGMP degradation by renal cGMP phosphodiesterase. The results provided herein demonstrate that PDE V inhibitor-treatment restored the renin and angiotensin inhibiting actions of exogenous BNP that was not observed in the untreated group.

In conclusion, in this model of experimental overt CHF, chronic administration of a PDE V inhibitor, Sildenafil, increased both plasma and urinary cyclic GMP with improved LV remodeling. Furthermore, it enhanced the renal response to acute SQ BNP without adverse cardiovascular hemodynamic effects. These results help demonstrate the therapeutic potential of a strategy to maximize cyclic GMP by combined PDE V inhibition and natriuretic peptides in CHF.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising an inhibitor of type V phosphodiesterase and a polypeptide having brain natriuretic peptide activity.

2. The composition of claim 1, wherein said inhibitor is sildenafil citrate.

3. The composition of claim 1, wherein said polypeptide is a human brain natriuretic peptide.

4. The composition of claim 1, wherein said polypeptide is nesiritide.

5. The composition of claim 1, wherein between about 1 mg and 100 mg of said composition is said inhibitor.

6. The composition of claim 1, wherein between about 20 mg and 75 mg of said composition is said inhibitor.

7. The composition of claim 1, wherein between about 200 μg and 20 mg of said composition is said polypeptide.

8. The composition of claim 1, wherein between about 500 μg and 10 mg of said composition is said polypeptide.

9. A method for reducing left ventricular mass in a mammal, said method comprising:
(a) identifying a mammal in need of reduced left ventricular mass, and
(b) administering an inhibitor of type V phosphodiesterase to said mammal under conditions wherein the left ventricular mass of said mammal decreases.

10. The method of claim 9, wherein said inhibitor is sildenafil citrate.

11. The method of claim 9, wherein said method comprises administering a polypeptide having brain natriuretic peptide activity to said mammal.

12. The method of claim 9, wherein said polypeptide is a human brain natriuretic peptide.

13. The method of claim 9, wherein said polypeptide is nesiritide.

14. A method for increasing the renal action of a polypeptide having brain natriuretic peptide activity, said method comprising administering an inhibitor of type V phosphodiesterase and said polypeptide to a mammal.

15. The method of claim 14, wherein said inhibitor is sildenafil citrate.

16. The method of claim 14, wherein said polypeptide is a human brain natriuretic peptide.

17. The method of claim 14, wherein said polypeptide is nesiritide.

18. The method of claim 14, wherein said mammal is a human.

19. The method of claim 14, wherein said mammal is at risk of experiencing heart failure or renal failure.

20. The method of claim 19, wherein said method comprises identifying said mammal has being at risk of experiencing heart failure or renal failure.
